(19) United States
(12) Patent Application Publication
(10) Pub. No.: US 2007/0204387 A1
(43) Pub. Date: Sep. 6, 2007

(54) Device for Dispensing a Fluid

(75) Inventors: Gay Joyce Cornelius, Cottingham (GB); Malcolm Tom McKechnie, Hull (GB)

Correspondence Address: NORRIS, MCLAUGHLIN & MARCUS, 875 THIRD AVE, 18TH FLOOR, NEW YORK, NY, 10022, US

(73) Assignee: RECKITT BENCKISER (UK) LIMITED, Slough, Berkshire (GB)

(21) Appl. No.: 10/597,297

(22) PCT Filed: Jan. 21, 2005
(86) PCT No.: PCT/GB05/00205

§ 371 Date: Aug. 30, 2006

(30) Foreign Application Priority Data
Jan. 23, 2004 (GB) 0401409.8

(51) Int. Cl. E03D 9/03; A61L 9/12

(52) U.S. Cl. 4/223

(57) ABSTRACT

A device for dispensing a fluid at a locus comprises a reservoir 2 of fluid in liquid form, a syphonic-action elongate liquid delivery means 12 having a proximal end 14 inside the reservoir adjacent the bottom thereof and a distal end 16 from which the fluid is dispensed as a liquid or as a vapour.

This invention relates to a device for dispensing a fluid. In particular, it relates to a device for delivering to a locus a liquid or vapour, for example a cleaning agent, a disinfecting agent, a deodorising agent, a fragrance, an insecticide, a miticide or an anti-allergenic agent.

In certain aspects the invention relates to the dispensing of a cleaning or disinfectant liquid into a toilet bowl or cistern, or a like vessel containing water or washed through with water.

Various toilet hygiene devices are known. Simple slow-dissolving disinfectant blocks are available, for placement in a cistern. Devices for releasing charges of a disinfectant or cleaning agent have been proposed, for placement in a cistern. Often such devices are over-complicated, and with any product placed in a cistern, a householder may forget about it or be slow to replace it when it has been exhausted.

Devices have also been proposed for clipping onto the rim of a toilet bowl, for releasing charges of a disinfectant or cleaning agent directly into the toilet bowl, on the squeezing action of the toilet seat. Such devices have not gained acceptance.

Devices are available, for clipping onto the rim of a toilet bowl, having disinfectant-containing “cages” suspended within the toilet bowl, and washed through with water when the toilet is flushed. Such devices are somewhat bulky and obtrusive within the toilet bowl, may themselves physically impede good cleaning of the toilet bowl by the flush water, and may be unpleasant to replace or replenish.

There is a need for a device which can deliver a liquid into a vessel over an extended period, which remains visible to a householder, but which does not require the presence of a bulky item within the vessel.

In certain aspects the invention relates to the dispensing of a vapour into an airspace.

Despite the plethora of devices available to dispense fragrances, insecticides and the like as vapours they all have drawbacks and there remains a need for a simple, reliable device for this purpose.

In accordance with a first aspect of the present invention there is provided a device for dispensing a fluid at a locus, the device comprising a reservoir of fluid in liquid form, a syphonic-action elongate liquid delivery means having a proximal end inside the reservoir adjacent the bottom thereof and a distal end from which the fluid is dispensed as a liquid or as a vapour.

The device may be one wherein the fluid is dispensed from the distal end as a liquid. Such a device may include an emanator wetted in use by liquid dispensed from the distal end.

A device of the present invention may be adapted for dispensing of a liquid into a vessel, the device having means for securing the device adjacent the rim of the vessel with the reservoir outside the vessel and with the distal end of the liquid delivery means inside the vessel.

The vessel may, for example, be the bowl of a toilet, typically having a generally horizontal rim.

Alternatively the vessel may be the cistern of a toilet.

When the vessel is a toilet bowl or cistern the liquid may be any liquid useful in fragrancing and/or disinfecting and/or cleaning and/or descaling of, and/or inhibiting the formation of scale in, the toilet bowl or cistern. The term “cleaning liquid” will be used herein to denote all such liquids.

A device in accordance with the invention may be used in other situations, in which it is desired to deliver a liquid inside a vessel, from a reservoir located outside the vessel.

For example, the device may be a convenient way of delivering an anti-corrosive liquid into the header tank of a central heating system. The invention can be used in domestic or industrial context.

Alternatively, the device may be a convenient way of delivering a treatment agent into a refuse container, e.g. such as a dustbin/garbage can (which could be located inside or outside a building). Preferred examples of treatment agents for this embodiment include fragrances, malodour treatment agents, biocides, bactericides and composters. The invention can be used in domestic or industrial context.

In a further alternative, the device may be a convenient way of delivering a cleaning agent into a vessel being used with a cleaning element, e.g. such as a mop and bucket. In this case the liquor inside the cleaning vessel may be at least partially revitalized with an active agent. Preferred examples of active agents for this embodiment include surface cleaning compositions, bleaches, polishes/waxes and surfactants. The invention can be used in domestic or industrial context. Similarly the device may be used to dispense an active into an automatic washing machine (e.g. such as a dishwasher) or an active (such as food or a cleaning agent) into a fish tank.

Preferably the means for securing the device adjacent to the rim of the vessel is able to secure the device onto the rim of the vessel. Suitably such means comprises a part which extends over the rim and a part which extends downwardly therefrom, into the vessel, in use. Preferably, the device defines a generally U-shaped opening adapted to engage resiliently over the rim, in use. One limb of the U-shaped opening may be defined, wholly or in part, by a wall of the reservoir.

Preferably, when the means for securing the device onto the rim of the vessel comprises a part which extends over the rim and a part which extends therefrom downwardly into the vessel, in use, the elongate liquid delivery means is in contact with each of the said parts.

In such embodiments the reservoir is preferably of somewhat flat shape, so that it projects only a short distance to the side of the rim of the vessel; thus, its thickness is preferably less than its height, and less than its width.

In other embodiments the device may be such that the fluid is dispensed from the distal end as a vapour, for example an insecticidal, insect-repellent, miticidal, deodorising, fragrancing or anti-allergenic vapour. The vapour may be directed to an emanator pad or emanator device.

The device may be such that a user initiates delivery but such that once delivery has started it continues without further action of the user.

In an alternative embodiment the device is self-initiating, after being removed from packaging and located in its working position. For example it may be sold as a pre-primed device. Delivery is commenced, for example, by the action of tearing off or piercing a foil, or foils, to expose the distal end, and a venting hole.

The device may include a means which can be operated by a person's hand to prime the syphonic-action delivery means, and thereby commence delivery. Typically such means will operate to reduce the volume of the reservoir.

Suitably venting means is provided to permit air to enter the vessel once dispensing commences.

The syphonic-action elongate liquid delivery means may be tubing, preferably plastics tubing. Preferably the internal cross-sectional area of the tubing does not exceed 12 mm². Preferably it does not exceed 5 mm². More preferably it does not exceed 2 mm². Most preferably it does not exceed 1 mm². Preferably its internal cross section is at least 0.1 mm², more preferably at least 0.3 mm², most preferably at least 0.5 m². Conveniently, it may have a circularly cylindrical bore.

The syphonic-action elongate liquid delivery means may be a capillary feed means, the capillary feed means preferably being of minimum cross-sectional area not exceeding 10 mm².

Suitably such a capillary feed means has one or more linear capillary pathways. Preferably the or each linear capillary pathway extends longitudinally along the capillary feed means (in other words, parallel to the axis of the capillary feed means). Preferably such a capillary feed means has an identical cross sectional shape, internally and externally, all along its length (a fibrous body does not have the same internal cross section all along its length, having regard to fibre or filament ends). Preferably, the or each linear capillary pathway has a cross-section of identical shape and size throughout its length.

However the cross-sectional area could in principle vary. When we refer to minimum cross-sectional area we refer to the smallest cross-section presented between the proximal and distal ends of a capillary feed means of variable cross section.

Suitably a capillary feed means having one or more linear capillary pathways is a longitudinally formed body. Preferably it is body formed by extrusion (including a body made of severe parallel extrusions coalesced together). Preferably it is formed of plastics material.

A capillary feed means may have one or more linear capillary pathway(s), for example longitudinal grooves or striations, formed at its external surface. For example, the external surface may be generally cylindrical, suitably circularly cylindrical, but superimposed on the cylindrical surface may be longitudinal grooves or striations. In another embodiment the capillary feed means may be polygonal in cross section, with each polygonal face and/or the longitudinal edges having longitudinal grooves or striations. When we refer to cross-sectional area herein we mean to include the area of such external grooves or striations. In practical terms the cross-sectional area of an externally indented capillary feed means may, for the purpose of this specification, be regarded as the area bounded by a length of filament wrapped around the capillary feed means.

Alternatively a capillary feed means may have one or more internal linear capillary pathway(s).

A capillary feed means may have one or more linear capillary pathway(s) formed at its external surface and one or more internal capillary pathway(s).

A preferred capillary feed means has one or more internal linear capillary pathway(s).

In other embodiments a capillary feed means may be of consolidated fibre form, for example of consolidated cellulose or plastics fibres. The fibres may be bonded together for rigidity. Suitable as a capillary feed means may, for example, be a fibrous body of the type used as an ink feeder and writing tip in “felt-tip” pens.

Preferably, a capillary feed means is of a material which is relatively rigid, and self-supporting when it is formed into a long thin body. By “relatively rigid” we can state the following. If one considers a capillary feed means held horizontally, with the distal end region projecting from the fingers, we consider it to be “relatively rigid” if it stays straight under its own weight, and does not bow or droop downwards more than 1 cm, when it projects 3 cm beyond the fingers. A preferred capillary feed means does not bow or droop downwards more than 1 cm when thus projected 5 cm, more preferably 10 cm, beyond the fingers, when thus held.

Suitable materials for a capillary feed means include plastics materials, including acrylics, polyamides, polyacetals (especially polyoxyethylene) and polyolefins (especially polypropylene), in the case of a longitudinally formed capillary feed means; and cellulose and polyester, in the case of a fibrous capillary feed means.

Preferably the minimum cross-sectional area of a capillary feed means is at least about 0.1 mm, more preferably at least about 0.18 mm², and most preferably at least about 0.25 mm².

Preferably the minimum cross-sectional area of a capillary feed means is not greater than about 8 mm², more preferably not greater than about 5 mm², most preferably not greater than about 4 mm². In the case of longitudinally formed capillary feed means the cross-sectional area may be not greater than about 2 mm², and, especially, not greater than 1 mm².

Preferably the diameter of a capillary feed means is in the range 0.1-3 mm, most preferably 0.5-2 mm. By “diameter” we mean the diameter itself when a capillary feed means is circularly cylindrical (as is preferred), and the maximum diameter, when it is not.

We have shown that by use of a suitable capillary feed means the device may be self-priming.

Suitably a capillary feed means may be encased in a plastics cover tube, but such that its distal end region is exposed.

Preferably the device is such that an operation a person undertakes to prime the syphonic-action liquid delivery means (when this is required) also opens venting means of the reservoir. For example one action by the user may reduce the volume of the reservoir to commence the delivery of the fluid and at the same time or immediately thereafter break a seal or otherwise vent the reservoir, which previously was sealed from the atmosphere.

The rate of delivery from the device can be determined by one or more of the following:
viscosity of the liquid
the pressure head
the venting airflow (which may be adjustable)
the size and design of the liquid delivery means.

The device is preferably of a form such that once its operation has commenced, it operates without any further agency of a person, but may be acted upon by a person to deliver an extra charge of the fluid.

The reservoir may have a flexible wall by means of which the reservoir can be compressed to prime the syphonic-action liquid delivery means and/or to deliver an extra charge of the fluid.

When the device is to be used for delivering a liquid into the bowl of a toilet the liquid delivery means may be provided with a means for preventing dripping of cleaning liquid from the distal end, into the bowl. Such means may be, for example, an absorbent part, for example a sintered plastics material, or fibrous material, or foam material. Alternatively the arrangement may be such that dripping takes place, but the liquid is either colourless, or is a colour which is acceptable to consumers, for example blue.

Such a part may also be used as a vapour emanator in other embodiments, in which a volatile liquid issues from the distal end of the liquid delivery means. In some embodiments such a part may line a partially open box within which the device is located, in use.

Preferably the liquid within the reservoir is of a type which does not evaporate to leave crystalline material or other solid residue. Preferably it does not dry out. Most preferably it contains a humectant.

Preferably, the reservoir is narrow at its bottom. More specifically, the horizontal cross section area of the reservoir is least adjacent to the bottom of the reservoir. Preferably, the reservoir tapers in a downward direction.

The reservoir may carry an external, removable, face plate. Face plates of alternative colours and/or designs may be available, so that a householder can choose a face plate which matches their decor.

In accordance with a second aspect of the invention there is provided a method of dispensing a fluid at a locus, using a device as defined above.

The invention will now be further described with reference to the accompanying drawings, in which:

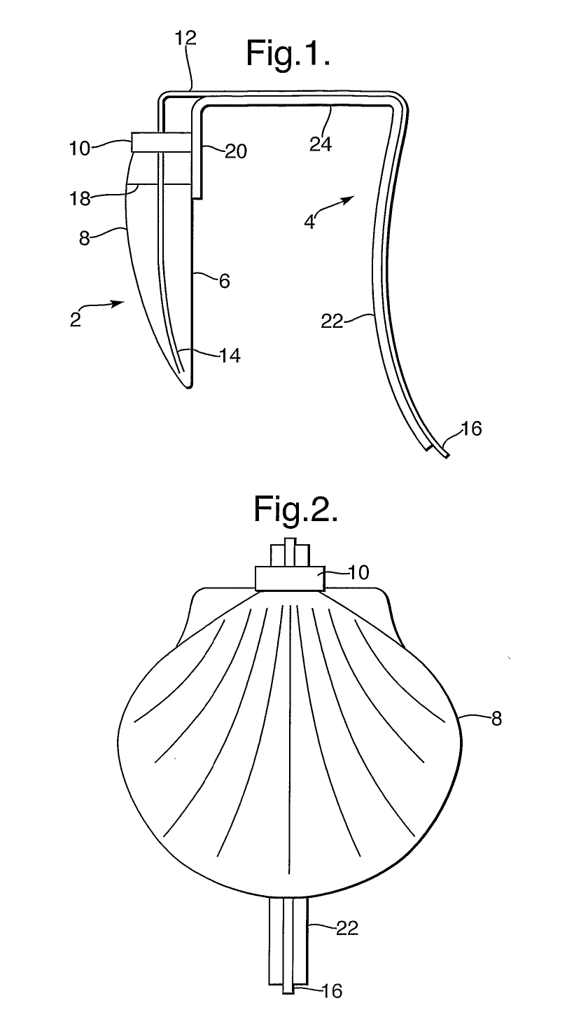

FIG. 1 shows, in schematic side cross sectional view, a device in accordance with the invention, for delivering cleaning liquid to the bowl of a toilet;

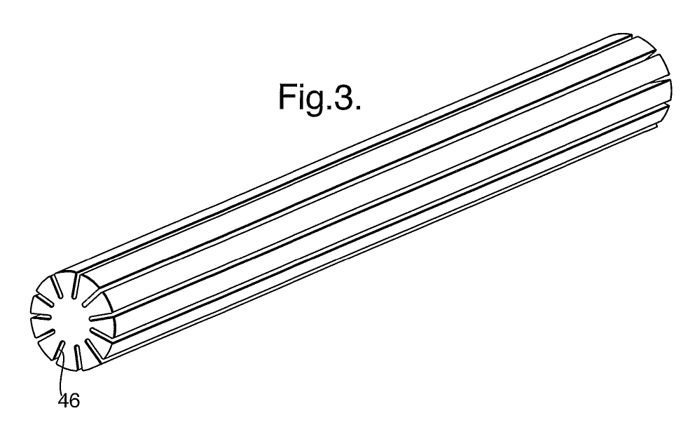

FIG. 2 shows, in front elevational view, the device of FIG. 1;

FIG. 3 shows, in perspective view, an alternative component for use in a device such as that shown in FIGS. 1 and;

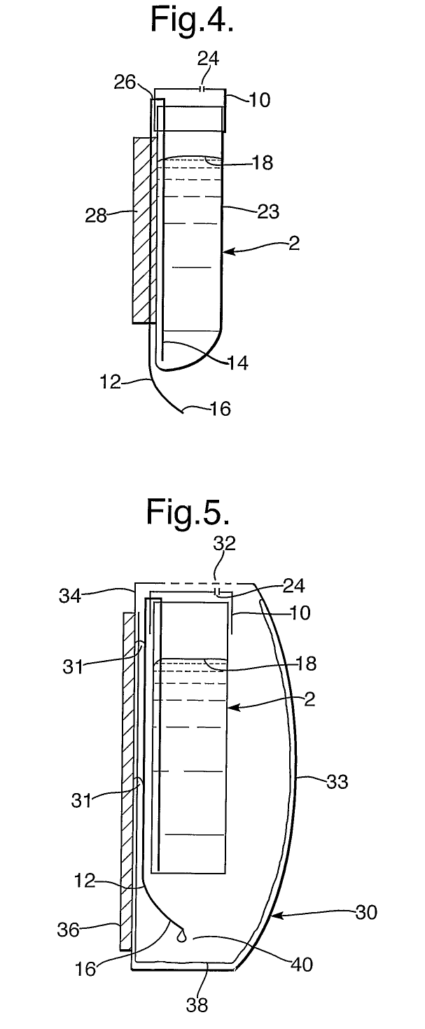

FIG. 4 shows, in schematic cross sectional view, a device in accordance with the invention, for dispensing a vapour into an air space; and

FIG. 5 shows in schematic cross sectional view, a further device in accordance with the invention, for dispensing a vapour into an air space.

The device shown in FIGS. 1 and 2 comprises a reservoir 2 and a flexible resilient clip formation generally indicated as 4, attached to the reservoir. The reservoir has a flat rear wall 6 which, in use, is adjacent to the rim of a toilet bowl, and a convex front wall 8, which curves around to meet the rear wall 6. The reservoir is sealed by an uppermost cap 10. A very thin tube 12 runs through the reservoir, through the cap 10, and then along the clip formation 4, being integrally formed with the clip formation or secured to the clip formation. The tube has a proximal end 14 at the bottom of the reservoir, and a distal end 16, which is below the end of the clip formation 4, and below the level of the bottom of the reservoir. The cap 10 is provided on its upper surface with a venting pinhole (not shown) which at the time of purchase is sealingly covered by a tear-off foil strip (not shown). The tube 12 passes through the cap in a sealing manner.

The reservoir is formed of a flexible plastics material. It contains a cleaning liquid, whose surface is indicated by numeral 18.

The reservoir tapers in the downward direction, being narrowest at the bottom, and the proximal end 14 of the tube is very close to the narrow bottom of the reservoir, in order to be able to deliver almost all of the cleaning liquid within the reservoir.

The clip formation has a shorter limb 20 secured to the upper region of the rear wall 6 of the reservoir, a longer limb 22 and, between limbs 20 and 22, a connecting web 24. The rear wall 6, limbs 20 and 22 and connecting web 24 thus define a generally U-shaped downward-facing opening, and by means of this opening the device is able to be easily secured over the rim of a toilet bowl. The limb 22 is somewhat curved, as shown in FIG. 1 in its rest position, being closest to the rear wall of the reservoir in its middle region, and furthest from the rear wall of the reservoir at its extremities. It is dimensioned such that a householder can fit the device to the rim of a toilet bowl by simple downward movement, during which the rim will engage with the limb 22 and cause it to flex somewhat away from the reservoir. When the rim of the toilet bowl touches the connecting web 24 the device has been fitted and in this position the limb 22 is still somewhat displaced from its rest position, thereby providing a retaining force.

The limbs 20, 22 and the connecting web 24 are in the form of a strip or band and are moulded together from a suitable plastics material.

Once the device has been located over the rim of a toilet bowl, delivery of the cleaning liquid is commenced as follows.

The flexible front wall 8 is pressed towards the rear wall 6 to reduce the volume of the reservoir. This drives some cleaning liquid through the tube and out of the distal end 16, and into the toilet bowl. The outer wall 8 is released and at the same time the tear-off foil is torn away to permit air to vent into the reservoir. By this means the tube is primed and continues to deliver cleaning fluid slowly and continuously, by a syphonic action. The rate of delivery of cleaning fluid is dependant upon several factors, including the height of the distal end 16 relative to the reservoir, the thickness of the tube, and the viscosity of the cleaning liquid. The rate of delivery of the cleaning liquid and the size of the reservoir are preferably selected, such that the continuous delivery takes place over an extended period, for example about 6-30 days, and preferably about 20 days, before the cleaning liquid is exhausted.

If wished the vent may be provided with a damper to alter the inward air flow rate. In other embodiments alternative vents may be provided, of different sizes, covered by respective tear-off foils. By such means the liquid feed rate may be selected.

If the user requires it they may at any time after commencement press the flexible front wall to deliver a discrete extra charge of cleaning liquid.

It will be appreciated that cleaning liquid will drip from the distal end 16. For cosmetic reasons, in this embodiment, the cleaning liquid is colourless.

The front wall 8 of the reservoir may be moulded with a decorative motif, for example depicting a shell, leaf or fish. In the embodiment shown it is moulded with a scallop shell motif Alternatively it may be of plain appearance but able to carry a removable face plate. In such embodiments face plates of alternative colours and/or designs are available, so that the householder is able to match the appearance of the device, to their decor.

In other embodiments the device may be such that the measure taken to commence the delivery of liquid may act both to drive fluid through the tube and, at the correct moment, to vent the reservoir to the atmosphere.

In other embodiments there may be provided means for preventing dripping from the distal end 16 and/or means for catching drips; for example a drip tray and/or an absorbent pad or block.

In one experiment the solution to be delivered was a 2% w/w solution of a sodium alginate, PROTANAL LF 20/40 available from FMC Biopolymer of Drammen, Norway. This was added to the deionized water, and stirred until the PROTANAL was in solution. Its viscosity was 889 mPas. 50 ml of the solution was poured into a reservoir. The internal diameter of the silicone plastics delivery tube was 1 mm and the initial pressure head—the difference in height between the liquid level and the tube outlet—was 90 mm. It was found that the reservoir took 163 hours to empty. In contrast, with deionized water—of viscosity 1 mPas—the reservoir took 20 minutes to empty.

In another experiment a 1% w/w solution of PROTANAL LF 20/40 was made as described above. Its viscosity was 117 mPas. A silicone plastics delivery tube of internal diameter 0.5 mm was used. When the initial pressure head was 150 mm the reservoir took 195 hours to empty. When the initial pressure head was 100 mm the reservoir took 15 days to empty.

A further embodiment of the invention intended for delivery of a cleaning liquid to a toilet cistern is the same as the device shown in FIGS. 1 and 2, except that the connecting web 24 is much shorter, because of the narrow rim of a typical cistern. In such an embodiment the cleaning liquid is preferably a coloured liquid, for example a blue liquid. In such an embodiment the tube, or other elongate delivery means, may have its distal end above the highest water level in the cistern (but below the bottom of the reservoir of the device); in which case it operates in the same manner as the device shown in FIGS. 1 and 2. Alternatively it may have its distal end immersed in the water in the cistern when the water is at its highest level, in which case delivery of cleaning fluid takes place at intervals, only when the toilet is flushed and the water level in the cistern drops, to below the distal end of the tube.

A further embodiment, also with a short connecting web 24, is to deliver a sanitising liquid progressively into a waste receptacle, for example a dustbin, a nappy bin, a kitchen bin or a bin intended for medical or veterinary waste products.

In a further embodiment a capillary tube is employed, instead of a very thin (but non-capillary) tube. The capillary tube used is that shown in perspective view in FIG. 3. It is generally circularly cylindrical, and has no internal capillary pathway or pathways. It is an extruded monofilament of a relatively stiff polyacetal material. It is available from Aubex Corporation of Japan. It has a series of external longitudinal capillary pathways 46 in the form of eleven grooves, whose depth is greater than their width, and through which liquid can efficiently drawn by capillary action. The diameter of the longitudinally formed body shown in FIG. 3 is 0.6 mm. The grooves are approximately 0.15 mm deep and approximately 0.04 mm wide. This capillary feed means was found to be self-priming and to be able to transfer all of a liquid from the reservoir to the vessel.

A further embodiment, shown in FIG. 4, also has a reservoir 2 of liquid, whose surface is shown as 18, but emits it as a vapour from the distal end of the elongate delivery means 12. The elongate delivery means is a capillary tube as shown in FIG. 3.

The reservoir 2 is of decorative appearance, for example having a frontal face 23 shaped to connote a fruit, when the fragrance is that of a fruit. The reservoir is surmounted by a cap 10 having a pinhole vent 24 exposed on peeling off a metal foil (not shown).

The capillary tube has a proximal end 14 within the reservoir 12 adjacent its base and a distal end 16 outside the reservoir, lower than its base. The distal end is curved forwardly so that the dispensing of vapour is not close to a surface on which the device is retained in use. The proximal and distal ends are the ends of respective, generally parallel, limbs. There is only a very short connecting web 26 between these limbs, this connecting web passing through a side wall of the cap 10. The limb outside the reservoir, terminating in the distal end, is thus located very close to the reservoir.

The distal end 16 of the capillary tube may be of plain form, as shown (that is, no different from any other portion of the capillary tube) or may terminate in an emanator from which the liquid evaporates, or it may drip liquid onto a separate emanator from which the liquid evaporates. A suitable emanator may for example comprise a sintered lo ceramic or plastics block, a fibrous wad, a fabric, or a foam pad, A suitable emanator may, for example, comprise a sintered ceramic or plastics block, a fibrous wad, a fabric, or a foam pad.

The rear face of the reservoir carries an adhesive pad 28, which overlies the outer limb of the capillary tube, with the outer limb nesting in a slit or groove formed in the pad.

The liquid in the FIG. 4 embodiment is of a type which can evaporate to yield a desired air-modifying vapour. The vapour may have insecticidal, insect-repelling, miticidal, anti-allergenic, deodorising or fragrancing properties, or any other desired air-modifying property. The liquid contains a humectant to prevent drying out at the distal end.

To use the device of FIG. 4 the user peels off the foil to expose the pinhole vent 24, pulls off a cap (not shown) overlying the distal end of the capillary tube, peels off a cellophane sheet (not shown) to expose an adhesive surface of a pad 28, and sticks the device onto a suitable surface with the distal end lowermost. The device is self-priming, due to the capillary action of the tube. As the liquid evaporates from the distal end more liquid is drawn from the reservoir, until the liquid is eventually exhausted.

The embodiment in FIG. 5 is the same as that of FIG. 4, with like parts being given like reference numbers, except for the features now mentioned. The reservoir is of plain form and is encased in a box 30 open only at its upper end, the opening 32 being schematically indicated in FIG. 5 by dotted lines. The reservoir is removably secured inside the box by means of brackets 31 from which the reservoir may be lifted, to effect replacement by a full reservoir. The box has a decorative front wall 33 and a rear wall 34 which has affixed to it an adhesive pad 36. The box is lined inside with an absorbent material 38, for example a fabric, or a fibre wad, or foam sheet. Whereas the FIG. 4 device is designed to dispense all of its liquid by vaporisation directly from the distal end of the capillary tube, the FIG. 5 device is designed to dispense its liquid both by vaporisation directly from the distal end and from the absorbent material, via liquid having dripped (as at 40) slowly from the distal end, onto the absorbent material.

1. A device for dispensing a fluid at a locus, the device comprising a reservoir of fluid in liquid form, a syphonic-action elongate liquid delivery means having a proximal end inside the reservoir adjacent the bottom thereof and a distal end from which the fluid is dispensed as a liquid or as a vapour.

2. A device according to claim 1, wherein the fluid is dispensed from the distal end as a liquid.

3. A device according to claim 2, wherein the device includes a part wetted in use by liquid dispensed from the distal end, from which part liquid or vapour passes to the locus to be treated.

4. A device according to claim 1, adapted for dispensing of a liquid into a vessel, the device having means for securing the device adjacent the rim of the vessel with the reservoir outside the vessel and with the distal end of the liquid delivery means inside the vessel.

5. A device according to claim 4, wherein the means for securing the device onto the rim comprises a part which extends over the rim of the vessel and a part which extends therefrom downwardly into the vessel, in use.

6. A device according to claim 5, wherein the liquid delivery means is in contact with each of the said parts.

7. A device according to claim 5, wherein the device defines a generally U-shaped opening adapted to engage resiliently over the rim, in use.

8. A lavatory sanitizing device adapted for securement onto the rim of a lavatory bowl according to claim 1.

9. A device according to claim 1, adapted for securement onto the rim of a lavatory cistern.

10. A device according to claim 1, wherein the fluid is dispensed from the distal end as a vapour.

11. A device according to claim 1, wherein the device includes a means which can be operated by a person's hand to prime the syphonic-action liquid delivery means.

12. A device according to claim 11, wherein the reservoir has a flexible wall by means of which the reservoir can be compressed to prime the syphonic-action liquid delivery means.

13. A device according to claim 10, wherein the device is self-priming.

14. A device according to claim 1, wherein the liquid delivery means is plastics tubing.

15. A device according to claim 1, wherein the liquid delivery means is a capillary feed means.

16. A device according to claim 1, wherein the device comprises an absorbent body onto which liquid passes from the distal end.

17. A device according to claim 1, wherein the liquid is of such a type that it does not dry out at the distal end to leave a solid residue.

18. A method of dispensing a fluid at a locus, using a device according to claim 1.

19. (canceled)
